(19) United States
(12) Patent Application Publication
(10) Pub. No.: US 2008/0081926 A1
(43) Pub. Date: Apr. 3, 2008

(54) SULFONYLAMINOBENZOIC ACID AND SALT

(75) Inventor: Joachim Brendel, Bad Vilbel (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/870,639

(22) Filed: Oct. 11, 2007

Related U.S. Application Data
(63) Continuation of application No. 11/355,303, filed on Feb. 15, 2006.
Application No. 11/355,303 is a continuation of application No. 10/796,466, filed on Mar. 9, 2004, now abandoned.
(60) Provisional application No. 60/492,637, filed on Aug. 5, 2003.

(30) Foreign Application Priority Data
Mar. 18, 2003 (DE) 10312073.4

(51) Int. Cl. C07C 311/01

(52) U.S. Cl. 562/430; 562/868

(57) ABSTRACT

The invention relates to a compound selected from the group consisting of 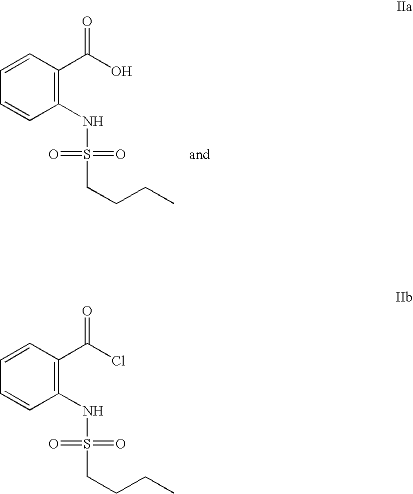 which is suitable for use in the synthesis of 2-(butyl-1-sulfonylamino)-N-[1(R)-(6-methoxypyridin-3-yl)propyl]benzamide.

### DESCRIPTION OF THE INVENTION

The invention relates to a sulfonylaminobenzoic acid and salt suitable for use in the synthesis of 2-(butyl-1-sulfonylamino)-N-[1(R)-(6-methoxypyridin-3-yl)propyl]benzamide of the formula I, and to the pharmaceutically tolerable salts of the benzamide, their preparation and use, in particular in medicaments.

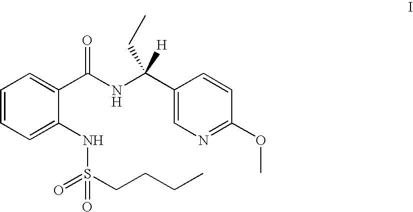

The compound of the formula I and its pharmaceutically tolerable salts can reduce the occurrence of atrial arrhythmias without an action on the heart chamber or other side effects occurring. The compound according to the invention and its pharmaceutically tolerable salts are therefore particularly suitable as a novel antiarrhythmic active compound, in particular for the treatment and prophylaxis of atrial arrhythmias, for example atrial fibrillation (AF) or atrial flutters.

Atrial fibrillation and atrial flutters are the most frequent, lasting cardiac arrhythmias. The occurrence increases with advancing age and frequently leads to fatal concomitant symptoms, such as, for example, cerebral infarct. AF affects about 1 million Americans yearly and leads to more than 80,000 strokes each year in the USA. The antiarrhythmics of classes I and III customary at present reduce the reoccurrence rate of AF, but are only used restrictively because of their potential proarrhythmic side effects. There is therefore a great medical need for the development of better medicaments for the treatment of atrial arrhythmias (S, Nattel, Am. Heart J. 130, 1995, 1094-1106; “Newer developments in the management of atrial fibrillation”).

It has been shown that most supraventricular arrhythmias are subject to “reentry” excitation waves. Such reentries occur when the cardiac tissue possesses a slow conductivity and at the same time very short refractory periods. The increase in the myocardial refractory period due to prolongation of the action potential is a recognized mechanism for ending arrhythmias or preventing their formation (T. J. Colatsky et al., Drug Dev. Res. 19, 1990, 129-140; “Potassium channels as targets for antiarrhythmic drug action”). The length of the action potential is essentially determined by the extent of repolarizing K⁺ currents which flow out of the cell via various K⁺ channels.

Particularly great importance is ascribed here to the “delayed rectifier” IK, which consists of 3 different components: IKᵣ, IKₛ and Ikᵤᵣ.

Most known class III antiarrhythmics (for example dofetilide, E4031 and d-sotalol) mainly or exclusively block the rapidly activating potassium channel IKᵣ, which can be detected both in cells of the human ventricle and in the atrium. It has been shown, however, that these compounds have an increased proarrhythmic risk at low or normal heart rates, arrhythmias, which are described as “torsades de pointes”, in particular being observed (D. M. Roden, Am. J. Cardiol. 72, 1993, 44B-49B; “Current status of class III antiarrhythmic drug therapy”). Beside this high and in some cases fatal risk at a low rate, a decrease in the activity under the conditions of tachycardia, in which the action is needed in particular, was found for the IKᵣ blockers (“negative use dependence”).

The “particularly rapidly” activating and very slowly inactivating component of the delayed rectifier Ikᵤᵣ (=ultra-rapidly activating delayed rectifier), which corresponds to the Kv10.5 channel, plays a particularly large part for the repolarization time in the human atrium. An inhibition of the IKᵤᵣ potassium outward current thus represents, in comparison to the inhibition of IKᵣ or IKₛ, a particularly effective method for the prolongation of the atrial action potential and thus for the ending or prevention of atrial arrhythmias.

In contrast to IKᵣ and IKₛ, which also occur in the human ventricle, the IKᵤᵣ in fact plays an important part in the human atrium, but not in the ventricle. For this reason, in the case of inhibition of the IKᵤᵣ current in contrast to the blockade of IKᵣ or IKₛ, the risk of a proarrhythmic action on the ventricle should be excluded from the start. (Z. Wang et al, Circ. Res. 73, 1993, 1061-1076: “Sustained Depolarisation-Induced Outward Current in Human Atrial Myocytes”; G.-R. Li et al., Circ. Res. 78, 1996, 689-696: “Evidence for Two Components of Delayed Rectifier K⁺ Current in Human Ventricular Myocytes”; G. J. Amos et al., J. Physiol. 491, 1996, 31-50: “Differences between outward currents of human atrial and subepicardial ventricular myocytes”).

Antiarrhythmics which act via a selective blockade of the IKᵤᵣ current or Kv1.5 channel have, however, not been available hitherto on the market.

The enantiomer 2-(butyl-1-sulfonylamino)-N-[1(R)-(6-methoxypyridin-3-yl)-propyl]benzamide claimed in this application has not been described hitherto. The corresponding racemate is mentioned as an example in the patent application WO 0288073. The compound of the formula I is distinguished by surprising advantages.

It has now surprisingly been found that the antiarrhythmic action for the 2-(butyl-1-sulfonylamino)-N-[1(R)-(6-methoxypyridin-3-yl)propyl]benzamide of the formula I according to the invention is excellent in a model on the anesthetized pig, while the corresponding 1(S) enantiomer is more weakly active. It has furthermore been found that the compound of the formula I has no effect on the QTc interval and no negative inotropic or hemodynamic side effects.

The experiments confirm that the compound I can be used as a novel antiarrhythmic having a particularly advantageous safety profile. In particular, the compound is suitable for the treatment of supraventricular arrhythmias, for example atrial fibrillation or atrial flutters. The compound can be employed for the termination of existing atrial fibrillation or flutters for the regaining of the sinus rhythm (cardioversion). Moreover, it reduces the susceptibility to the formation of new fibrillation events (retention of the sinus rhythm, prophylaxis).

The present invention relates to 2-(butyl-1-sulfonylamino)-N-[1(R)-(6-methoxypyridin-3-yl)propyl]benzamide of the formula I, and to its pharmaceutically acceptable salts.

Since the compound I contains a basic pyridine radical, it can also be used in the form of its pharmaceutically tolerable acid addition salts with inorganic or organic acids, for example as a hydrochloride, phosphate, sulfate, methanesulfonate, acetate, lactate, maleate, fumarate, malate, gluconate etc. The sulfonamide group present moreover makes possible the formation of alkali metal or alkaline earth metal salts, preferably the sodium or potassium salt, or ammonium salts, for example salts with organic amines or amino acids. The pharmaceutically tolerable salts can be obtained from the compound of the formula I by customary processes, for example by combination with an acid or base in a solvent or dispersant or alternatively from other salts by anion or cation exchange.

The free compound 2-(butyl-1-sulfonylamino)-N-[1(R)-(6-methoxypyridin-3-yl)propyl]benzamide of the formula I is preferred.

The compound of the formula I can be prepared by different chemical processes, of which two preparation possibilities are outlined in scheme 1. The coupling of the sulfonylaminobenzoic acid of the formula II with the amine of the formula III can be carried out either directly from the acid in the presence of a customary coupling reagent, or, for example, from an activated acid derivative such as the acid chloride. When using racemic 1-(6-methoxypyridin-3-yl)propylamine of the formula III, the cleavage into the enantiomers takes place in the final stage, for example by chiral chromatography or conventional resolution. Alternatively, the desired enantiomer can be obtained directly by use of 1(R)-(6-methoxypyridin-3-yl)-propylamine of the formula IIIa. The sulfonylaminobenzoic acid of the formula IIa is prepared in a manner known to the person skilled in the art from the commercially obtainable substances aminobenzoic acid and butylsulfonyl chloride.

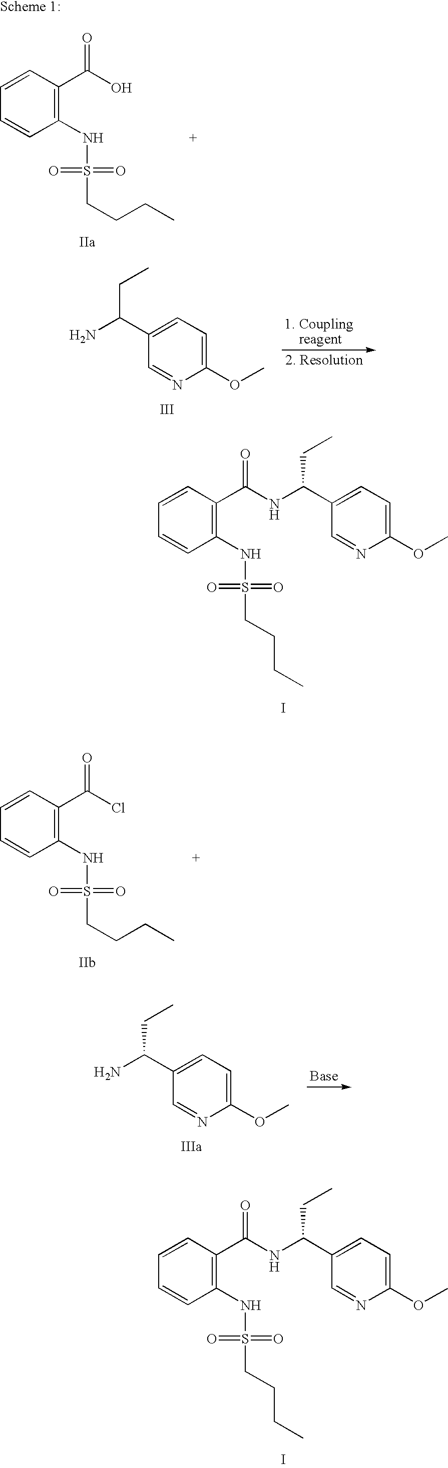

This application likewise includes the compound 1-(6-methoxypyridin-3-yl)-propylamine of the formula III employed as an intermediate, and its enantiomers, in particular 1(R)-(6-methoxypyridin-3-yl)propylamine of the formula IIIa, and its use for the preparation of pharmaceutical active compounds, for example of 2-(butyl-1-sulfonylamino)-N-[1(R)-(6-methoxy-pyridin-3-yl)propyl]benzamide.

1-(6-Methoxypyridin-3-yl)propylamine of the formula III can be prepared from commercially obtainable compounds by different chemical processes, of which two preparation possibilities are outlined as examples in scheme 2. On the one hand, 5-bromo-2-methoxypyridine can firstly be metalated using butyllithium, then reacted with propionitrile and subsequently reduced to the compound of the formula III using sodium borohydride. Alternatively, 3-cyano-6-methoxypyridine can be reacted with ethylmagnesium bromide and then reduced using sodium borohydride. The cleavage into the enantiomers can be carried out by customary methods, such as, for example, chromatography on a chiral phase, conventional resolution with the aid of a chiral acid or by enzymatic methods.

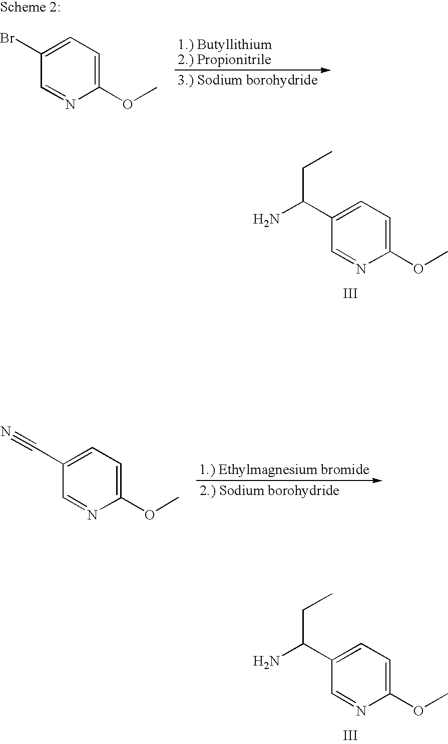

The compound of the formula I according to the invention and its physiologically tolerable salts can be used on animals, preferably on mammals, and in particular on humans, as a medicament on its own or in the form of pharmaceutical preparations. The present invention also relates to the compound of the formula I and its physiologically tolerable salts for use as a pharmaceutical, its use in the therapy and prophylaxis of cardiac arrhythmias, of supraventricular arrhythmias, of atrial fibrillation and/or atrial flutters and its use for the production of medicaments therefor. Furthermore, the present invention relates to pharmaceutical preparations which as active constituent contain an efficacious dose of the compound of the formula I and/or a physiologically tolerable salt thereof in addition to customary, pharmaceutically innocuous vehicles and excipients. The pharmaceutical preparations normally contain 0.1 to 90 percent by weight of the compound of the formula I and/or its physiologically tolerable salts. The pharmaceutical preparations can be produced in a manner known to the person skilled in the art. For this, the compound of the formula I and/or its physiologically tolerable salts, together with one or more solid or liquid pharmaceutical vehicles and/or excipients and, if desired, in combination with other pharmaceutical active compounds, are brought into a suitable administration form or dosage form, which can then be used as a pharmaceutical in human medicine or veterinary medicine.

Pharmaceuticals which contain the compound of the formula I according to the invention and/or its physiologically tolerable salts can be administered, for example, orally, parenterally, e.g. intravenously, rectally, by inhalation or topically, the preferred administration being dependent on the individual case, for example the particular clinical picture of the disease to be treated.

The person skilled in the art is familiar on the basis of his/her expert knowledge with excipients which are suitable for the desired pharmaceutical formulation. In addition to solvents, gel-forming agents, suppository bases, tablet excipients and other active compound carriers, it is possible to use, for example, antioxidants, dispersants, emulsifiers, antifoams, taste corrigents, preservatives, solubilizers, agents for achieving a depot effect, buffer substances or colorants.

To achieve an advantageous therapeutic action, the compound of the formula I can also be combined with other pharmaceutical active compounds. Thus, in the treatment of cardiovascular diseases advantageous combinations with substances having cardiovascular activity are possible. Possible combination partners of this type advantageous for cardiovascular diseases are, for example, other antiarrhythmics, that is class I, class II or class III antiarrhythmics, such as, for example, IKₛ or IKᵣ channel blockers, for example dofetilide, or furthermore hypotensive substances such as ACE inhibitors (for example enalapril, captopril, ramipril), angiotensin antagonists and K⁺ channel activators, and alpha-receptor blockers, but also sympathomimetic compounds and compounds having adrenergic activity, and Na⁺/H⁺ exchange inhibitors, calcium channel antagonists, phosphodiesterase inhibitors and other substances having positive inotropic activity, such as, for example, digitalis glycosides, or diuretics.

For an oral administration form, the active compound is mixed with the additives suitable therefor, such as vehicles, stabilizers or inert diluents, and brought by means of the customary methods into the suitable administration forms, such as tablets, coated tablets, hard gelatin capsules, aqueous, alcoholic or oily solutions. The inert carriers which can be used are, for example, gum arabic, magnesia, magnesium carbonate, potassium phosphate, lactose, glucose or starch, in particular corn starch. The preparation can be carried out here both as dry and moist granules. Suitable oily vehicles or solvents are, for example, vegetable or animal oils, such as sunflower oil or cod-liver oil. Suitable solvents for aqueous or alcoholic solutions are, for example, water, ethanol or sugar solutions or mixtures thereof. Further excipients, also for other administration forms, are, for example, polyethylene glycols and polypropylene glycols.

For subcutaneous, intramuscular or intravenous administration, the active compound, if desired with the substances customary therefor such as solubilizers, emulsifiers or further excipients, is brought into solution, suspension or emulsion. The compound of the formula I and its physiologically tolerable salts can also be lyophilized and the lyophilizates obtained used, for example, for the production of injection or infusion preparations. Suitable solvents are, for example, water, physiological saline solution or alcohols, for example ethanol, propanol, glycerol, in addition also sugar solutions such as glucose or mannitol solutions, or alternatively mixtures of the various solvents mentioned.

Suitable pharmaceutical formulations for administration in the form of aerosols or sprays are, for example, solutions, suspensions or emulsions of the active compound of the formula I or its physiologically tolerable salts in a pharmaceutically innocuous solvent, such as, in particular, ethanol or water, or a mixture of such solvents. If required, the formulation can also additionally contain other pharmaceutical excipients such as surfactants, emulsifiers and stabilizers, and a propellant. Such a preparation customarily contains the active compound in a concentration of approximately 0.1 to 10, in particular of approximately 0.3 to 3, percent by weight.

The dose of the active compound of the formula I or of the physiologically tolerable salts thereof to be administered depends on the individual case and is to be adapted to the conditions of the individual case as customary for an optimum action. Thus, it depends, of course, on the frequency of administration but also on the nature and severity of the illness to be treated and on the sex, age, weight and individual responsiveness of the human or animal to be treated and on whether the therapy is to be acute or chronic or prophylaxis is to be carried out. Customarily, the daily dose of the compound of the formula I in the case of administration to a patient weighing approximately 75 kg is 0.01 mg/kg of bodyweight to 100 mg/kg of bodyweight, preferably 0.1 mg/kg of bodyweight to 20 mg/kg of bodyweight. The dose can be administered in the form of an individual dose or divided into a number of, for example two, three or four, individual doses. In particular in the treatment of acute cases of cardiac arrhythmias, for example in an intensive care unit, parenteral administration by injection or infusion, for example by an intravenous continuous infusion, can also be advantageous.

### EXPERIMENTAL SECTION

### Preparation of 2-(butyl-1-sulfonylamino)-N-[1(R)-(6-methoxypyridin-3-yl)-propyl]benzamide

### a) 2-(Butyl-1-sulfonylamino)benzoic acid

20 g (188 mmol) of sodium carbonate were added to a suspension of 20 g (146 mmol) of 2-aminobenzoic acid in 250 ml of water. 11.4 g (72.8 mmol) of butylsulfonyl chloride were then added dropwise and the reaction mixture was stirred at room temperature for 2 days. It was acidified with concentrated hydrochloric acid, stirred at room temperature for 3 hours and the precipitated product was filtered off with suction. After drying in vacuo, 9.6 g of 2-(butyl-1-sulfonylamino)benzoic acid were obtained.

### b) 1-(6-Methoxypyridin-3-yl)propylamine

Method 1

3 ml (23.2 mmol) of 5-bromo-2-methoxypyridine were added at −70° C. to a solution of 10.2 ml of n-butyllithium (2.5 M solution in hexane; 25.5 mmol) in 50 ml of diethyl ether. After 10 min, 1.4 ml (19.5 mmol) of propionitrile were added. After 2 hours at −70° C., the reaction mixture was slowly allowed to come to room temperature. 2.2 g of sodium sulfate decahydrate were then added and the mixture was allowed to stir for 1 hour. After subsequent addition of 5 g of magnesium sulfate, the salts were filtered off after brief stirring and the filtrate was concentrated. The residue was dissolved in 70 ml of methanol and 1.1 g (28 mmol) of sodium borohydride were added at 0° C. After stirring overnight, the reaction mixture was adjusted to pH 2 using concentrated hydrochloric acid and concentrated on a rotary evaporator. The residue was treated with 10 ml of water and extracted once with diethyl ether. The aqueous phase was then saturated with sodium hydrogen carbonate, concentrated in vacuo and the residue was extracted with ethyl acetate. After drying and concentrating the ethyl acetate extracts, 1.4 g of racemic 1-(6-methoxypyridin-3-yl)propylamine were obtained.

The enantiomers were separated by preparative HPLC on a Chiralpak ADH column (250×4.6 mm); eluent: heptane/ethanol/methanol 50:1:1 with 0.1% of diethylamine; temperature: 30° C.; flow rate 1 ml/min.

First, 0.45 g of 1(S)-(6-methoxypyridin-3-yl)propylamine was eluted at a retention time of 18.4 min. 0.42 g of 1(R)-(6-methoxypyridin-3-yl)propyl-amine was then obtained at a retention time of 21.0 min.

Method 2

170 ml (170 mmol) of a 1 M solution of ethylmagnesium bromide in tetrahydrofuran were added dropwise at 0° C. under argon in the course of 30 minutes to a solution of 20 g (150 mmol) of 6-methoxynicotinonitrile and 0.62 g (3.3 mmol) of copper(I) iodide in 125 ml of anhydrous tetrahydrofuran. After 30 minutes, the reaction mixture was allowed to come to room temperature and was stirred for a further 3 h. 200 ml of methanol were then added dropwise at 5-10° C. and 11.3 g (299 mmol) of sodium borohydride were then added in portions. After stirring overnight at room temperature, 300 ml of water were added and the mixture was extracted 3 times using 250 ml of ethyl acetate each time. The organic phase was dried over magnesium sulfate, then concentrated and the residue was purified by chromatography. 5.5 g of racemic 1-(6-methoxypyridin-3-yl)propylamine were obtained.

### c) 2-(Butyl-1-sulfonylamino)-N-[1(R)-(6-methoxypyridin-3-yl)propyl]benz-amide and 2-(butyl-1-sulfonylamino)-N-[1(S)-(6-methoxypyridin-3-yl)propyl]-benzamide

Method 1

4.4 g (32.7 mmol) of 1-hydroxy-1H-benzotriazole and 6.3 g (32.7 mmol) of N-ethyl-N′-(3-dimethylaminopropyl)carbodiimide hydrochloride were added to a solution of 8.0 g (31.1 mmol) of 2-(butyl-1-sulfonylamino)benzoic acid in 250 ml of tetrahydrofuran and the reaction mixture was stirred for 90 min. A solution of 5.4 g (32.7 mmol) of racemic 1-(6-methoxypyridin-3-yl)propyl-amine in 20 ml of tetrahydrofuran was then added dropwise and the mixture was stirred overnight. The reaction mixture was treated with 250 ml of water and extracted with 300 ml of ethyl acetate. The organic phase was extracted 5 times with 100 ml of saturated sodium hydrogen carbonate solution each time and then dried over magnesium sulfate. 9.0 g of 2-(butyl-1-sulfonylamino)-N-[1-(6-methoxypyridin-3-yl)propyl]benzamide were obtained.

The enantiomers were separated by preparative HPLC on a Chiralpak ADH column (250×4.6 mm); eluent: heptane/ethanol/methanol 10:1:1; temperature: 30° C.; flow rate 1 ml/min.

First, 4.0 g of 2-(butyl-1-sulfonylamino)-N-[1(R)-(6-methoxypyridin-3-yl)-propyl]benzamide were eluted at a retention time of 5.9 min. After a mixed fraction, 3.0 g of 2-(butyl-1-sulfonylamino)-N-[1(S)-(6-methoxypyridin-3-yl)-propyl]benzamide were then obtained at a retention time of 7.2 min.

Method 2

0.9 g of 2-(butyl-1-sulfonylamino)-N-[1(R)-(6-methoxypyridin-3-yl)propyl]-benzamide was obtained from 0.41 g (2.46 mmol) of 1(R)-(6-methoxy-pyridin-3-yl)propylamine and 0.64 g (2.47 mmol) of 2-(butyl-1-sulfonylamino)-benzoic acid by coupling in the presence of 1-hydroxy-1H-benzotriazole and N-ethyl-N′-(3-dimethylaminopropyl)carbodiimide hydrochloride analogously to method 1.

### d) 2-(Butyl-1-sulfonylamino)-N-[1(R)-(6-methoxypyridin-3-yl)propyl]benzamide

2 g of the 2-(butyl-1-sulfonylamino)-N-[1(R)-(6-methoxypyridin-3-yl)propyl]-benzamide obtained according to method 1 or method 2 were dissolved in 9 ml of isopropanol in the presence of heat, then 8 ml of warm water were added and the reaction mixture was allowed to cool slowly overnight. After filtering off with suction at 0° C., 1.5 g of 2-(butyl-1-sulfonylamino)-N-[1(R)-(6-methoxypyridin-3-yl)propyl]benzamide were obtained as colorless needle-shaped crystals; melting point 97° C. The absolute configuration was confirmed from suitable monocrystals by x-ray structural analysis.

Pharmacological Investigations

Kv1.5 channels from humans were expressed in Xenopus oocytes. For this, oocytes were first isolated from Xenopus laevis and defolliculated. RNA encoding Kv1.5 and synthesized in vitro was then injected into these oocytes. After Kv1.5 protein expression for 1-7 days, Kv1.5 currents were measured at the oocytes using the two microelectrode voltage clamp technique. The Kv1.5 channels were in this case as a rule activated using voltage jumps to 0 mV and 40 mV lasting 500 ms. The bath was rinsed using a solution of the following composition: NaCl 96 mM, KCl 2 mM, CaCl₂ 1.8 mM, MgCl₂ 1 mM, HEPES 5 mM (titrated to pH 7.4 using NaOH). These experiments were carried out at room temperature. The following were employed for data acquisition and analysis: Geneclamp amplifier (Axon Instruments, Foster City, USA) and MacLab D/A converter and software (ADInstruments, Castle Hill, Australia).

The substances according to the invention were tested by adding them to the bath solution in different concentrations. The effects of the substances were calculated as percentage inhibition of the Kv1.5 control current which was obtained when no substance was added to the solution. The data were then extrapolated using the Hill equation in order to determine the inhibitory concentrations IC₅₀ for the respective substances.

In this manner, the following IC₅₀ values were determined for the compounds listed below:
2-(Butyl-1-sulfonylamino)-N-[1-(6-methoxypyridin-3-yl)propyl]benzamide: IC50=2.4 μM
2-(Butyl-1-sulfonylamino)-N-[1(R)-(6-methoxypyridin-3-yl)propyl]benzamide of the formula I:
   IC50=10 μM
2-(Butyl-1-sulfonylamino)-N-[1(S)-(6-methoxypyridin-3-yl)propyl]benzamide:
   IC50=2.4 μM

Investigation of the refractory period and the left-atrial vulnerability in the pig

The two enantiomers 2-(butyl-1-sulfonylamino)-N-[1(R)-(6-methoxypyridin-3-yl)propyl]benzamide of the formula I and 2-(butyl-1-sulfonylamino)-N-[1(S)-(6-methoxypyridin-3-yl)propyl]benzamide were investigated and compared for prolongation of the refractory period and antiarrhythmic activity on the atrium of the anesthetized pig. In the course of this, the refractory period of the left atrium was determined and the antiarrhythmic activity was recorded as described in the literature (Knobloch et al. 2002. Naunyn-Schmiedberg's Arch. Pharmacol. 366; 482-487). The anti-arrhythmic action relates here to the inhibition of the occurrence of episodes of arrhythmias which are induced by a prematurely placed extra-stimulus (S2) in the left atrium (=left-atrial vulnerability).

A comparison of the action of 2-(butyl-1-sulfonylamino)-N-[1(R)-(6-methoxypyridin-3-yl)propyl]benzamide of the formula I and 2-(butyl-1-sulfonylamino)-N-[1(S)-(6-methoxypyridin-3-yl)propyl]benzamide on the refractory period of the left atrium and antiarrhythmic activity in the anesthetized pig after a bolus administration of 3 mg/kg is shown in table 1. The refractory period values are stated in percent of the basal values 10 minutes after injection. Mean values for the refractory periods are shown from three rates (150, 200 and 250/min). From the results compiled in table 1, it is seen that the R enantiomer causes a markedly greater prolongation of the refractory period than the S enantiomer. By using the R enantiomer, it was possible to prevent 73.9% of the induced arrhythmias, while when using the S enantiomer the occurrence of arrhythmias was inhibited only by 27%.

TABLE 1

| | S enantiomer | | R enantiomer | |
|---|---|---|---|---|
| | Mean | | Mean | |
| | value | SEM | value | SEM |
| % increase in the refractory period | 8.8% | 3.4% | 19% | 4% |
| % inhibition of the arrhythmias | 27.3% | 2.4% | 73.9% | 11% |
| | n = 4 | | n = 6 | |

By repeated measurement after substance administration, the duration of action of a substance on the refractory period can be determined in this experimental procedure. The R enantiomer was infused intravenously over the course of 100 minutes in a dose of 1 mg/kg and the pharmacological action was determined over the course of 280 minutes. As shown in FIG. 1, 2-(butyl-1-sulfonylamino)-N-[1(R)-(6-methoxypyridin-3-yl)propyl]benzamide led to a long-lasting action on the left-atrial refractory period, which also continued unchanged for 180 minutes after ending the infusion.

### DESCRIPTION OF THE DRAWINGS

The following captions and markings were made in the drawing:

FIG. 1: duration of action on the refractory period of the left atrium of 2-(butyl-1-sulfonylamino)-N-[1(R)-(6-methoxypyridin-3-yl)propyl]benzamide, 1 mg/kg as an infusion over the course of 100 minutes intravenously

Y axis: % of the basal refractory period

X axis: time in minutes

2-(Butyl-1-sulfonylamino)-N-[1(R)-(6-methoxypyridin-3-yl)propyl]benzamide

Control without active substance.

What is claimed is:

1. A compound selected from the group consisting of [chemical structure]
